(19) United States
(12) Patent Application Publication
(10) Pub. No.: US 2016/0346453 A1
(43) Pub. Date: Dec. 1, 2016

(54) VASCULAR DEVICE

(71) Applicant: Rex Medical, L.P., Conshohocken, PA (US)

(72) Inventors: JAMES F. MCGUCKIN, JR., Radnor, PA (US); Stephan A. DeFonzo, Wayne, PA (US)

(73) Assignee: Rex Medical, L.P., Conshohocken, PA (US)

(21) Appl. No.: 15/095,048

(22) Filed: Apr. 9, 2016

Related U.S. Application Data
(60) Provisional application No. 62/168,740, filed on May 30, 2015.

(51) Int. Cl. A61M 1/36; A61B 17/12

(52) U.S. Cl. CPC A61M 1/3655; A61B 17/12109; A61B 17/12131

(57) ABSTRACT

A vascular device for reducing blood flow in a first vessel or a first graft to enable increased blood flow in a second vessel or second graft. The vascular device includes a support structure having a proximal portion, a distal portion and an intermediate portion, the support structure movable from a reduced profile insertion position to an expanded placement position. A covering material is supported by the structure, the intermediate portion in the expanded position having a transverse dimension less than a transverse dimension of the proximal and distal portions to restrict blood flow through the device and thereby increase blood flow to the second vessel or second graft. A method or reducing blood flow in one vessel or graft and increasing blood flow in another vessel or graft is also disclosed.

### BACKGROUND

This application claims priority from provisional application 62/168,740, filed May 30, 2015, the entire contents of which are incorporated herein by reference.

### TECHNICAL FIELD

This application relates to a vascular device and more particularly to a vascular device to restrict blood flow in a graft or vessel to thereby force increased blood flow to another vessel.

### BACKGROUND OF RELATED ART

The circulatory system is responsible for the circulation of blood through the vessels to transport oxygen and nutrients to the body's cells and carry carbon dioxide back to the lungs.

An arteriovenous fistula is an abnormal connection between an artery and a vein. Normally, blood flows from the arteries to capillaries and to the veins. Oxygen and nutrients in the blood travel from the capillaries to tissues in the body. With an arteriovenous fistula, blood instead flows directly from an artery into a vein, bypassing some capillaries. Bypassing of the capillaries can cause tissues below the capillaries to receive a diminished blood supply.

In vascular access steal syndrome, also referred to as dialysis associated steal syndrome, ischemia occurs as the blood supply to tissues is restricted causing a shortage of oxygen supply to the cells. In cases of limb ischemia, tissue necrosis, gangrene and paralysis can occur. There are numerous causes of inadequate blood flow to a body part, and it is imperative that adequate blood flow be restored.

In certain instances inside an arteriovenous fistula, too much flow causes steal syndrome, depriving other cells of needed blood and oxygen. This can occur as to much blood flow occurs thought the graft, which is connecting the artery to the vein, thereby depriving parts of the body of sufficient blood flow. Such stealing syndrome can also occur in a transplanted organ, in an artery associated with an arteriovenous malformation causing a high flow rate in a left to right shunt and in various bifurcated vessels. Such areas of deprived flow can include the extremities of the patient such as the hands and feet.

It would be advantageous to provide a system to restrict blood flow in a vessel or graft to thereby force more blood flow to areas of the body deprived of such flow. It would further be advantageous to provide such a system that achieves this in a minimally invasive manner to reduce risk and trauma to the patient and as well as reduce patient recovery time.

### SUMMARY

The present invention provides a vascular device and minimally invasive method to restrict blood flow in a vessel or graft thereby automatically increasing blood flow in another vessel or graft. Such vascular device can help treat ischemia by providing needed blood and oxygen to the flow deprived body part. The device can be utilized in branching vessels to increase flow to the other branching vessel. The device can also be used in arteriovenous fistulas to decrease blood flow through the graft and to increase blood flow through the artery.

In accordance with one aspect of the present invention, a vascular device is provided for reducing blood flow in a first vessel or a first graft to thereby increase blood flow in a second vessel or second graft. The vascular device comprises a support structure having a proximal portion, a distal portion and an intermediate portion, the support structure movable from a reduced profile insertion position to an expanded placement position. A covering material is supported by the support structure. The intermediate portion in the expanded position has a transverse dimension less than a transverse dimension of the proximal portion and the distal portion to restrict blood flow through the device and thereby increase blood flow to the second vessel or second graft.

In some embodiments, the device has a substantially hourglass shape.

In some embodiments, the device includes a first plurality of penetrating members at the proximal portion and a second plurality of penetrating members at the distal portion.

The intermediate portion in some embodiments includes a tubular portion. The tubular portion can have a plurality of elongated slots formed therein. The device, i.e., support structure, can include a first plurality of struts extending from and flaring out from the tubular portion in a distal direction and a second plurality of struts extending from and flaring out from the tubular portion in a proximal direction. In some embodiments, the first and second plurality of struts terminate in hooks. In some embodiments, the first and second plurality of struts are twisted to alter a plane of the hooks.

In some embodiments, the covering material is attached to an outer surface of the support structure. The covering material can alternatively be attached to an inner surface of the support structure.

In accordance with another aspect of the present invention, a method of restricting blood flow in a first lumen and increasing blood flow in a second lumen is provided. The method comprises providing a vascular device having an intermediate portion having a reduced transverse dimension and flared distal and proximal portions. The method further includes the step of placing the device in the first lumen, the reduced dimension intermediate portion reducing blood flow by providing a choke point and thereby increasing blood flow to the second lumen.

The first and/or second lumen can be a vessel or graft.

The method can further include the step of inserting a delivery sheath containing the vascular device and removing the delivery sheath to enable the vascular device to expand against the wall of the first lumen. In some embodiments, the vascular device is made of shape memory material and the step of removing the sheath to enable the vascular device to expand causes the vascular device to automatically expand against the wall of the lumen. The method in certain embodiments can further include the step of inserting a guidewire and inserting the delivery sheath over the guidewire.

In accordance with another aspect of the present invention, a method of increasing blood flow in a vessel in an arteriovenous fistula is provided. The method comprises the steps of providing a vascular device having a reduced transverse dimension intermediate portion and flared distal and proximal portions and placing the device in a lumen of a graft within a patient, the reduced dimension intermediate portion reducing blood flow in the lumen of the graft by providing a choke point and thereby increasing blood flow to the lumen of the vessel.

In the foregoing methods, the device, i.e., support structure, can be composed of shape memory material to expand automatically due to exposure to body temperature as in some embodiments it has a memorized configuration in the expanded state. Alternatively, removal of a sheath can enable the vascular device to expand because the sheath will no longer block expansion and a balloon catheter can be utilized wherein the balloon is inserted within the vascular device and inflated to radially expand the vascular device to engage the vessel walls.

### BRIEF DESCRIPTION OF THE DRAWINGS

Preferred embodiment(s) of the present invention are described herein with reference to the drawings wherein:

FIG. 1 is a perspective view of the vascular device of the present invention shown in the collapsed position;

FIG. 2 is a perspective view of the vascular device of FIG. 1 shown in the expanded position;

FIG. 2A is a side view of the vascular device of FIG. 1 with the covering removed for clarity to show the underlying support structure;

FIG. 3 is a side view of the vascular device of FIG. 1;

FIGS. 4A-4D are side views illustrating delivery of the vascular device of FIG. 1 to a branch of a bifurcated main vessel for placement downstream of the juncture, wherein:

FIG. 4A illustrates the delivery sheath for the vascular device inserted into a main vessel;

FIG. 4B illustrates the delivery sheath directed into a branch vessel;

FIG. 4C illustrates the vascular device delivered from the delivery sheath into a branch vessel; and

FIG. 4D illustrates withdrawal of the delivery sheath leaving the vascular device in the expanded position within the branch vessel.

FIG. 5 is a side view illustrating the vascular device of the present invention placed in a graft of an arteriovenous fistula.

### DETAILED DESCRIPTION OF PREFERRED EMBODIMENTS

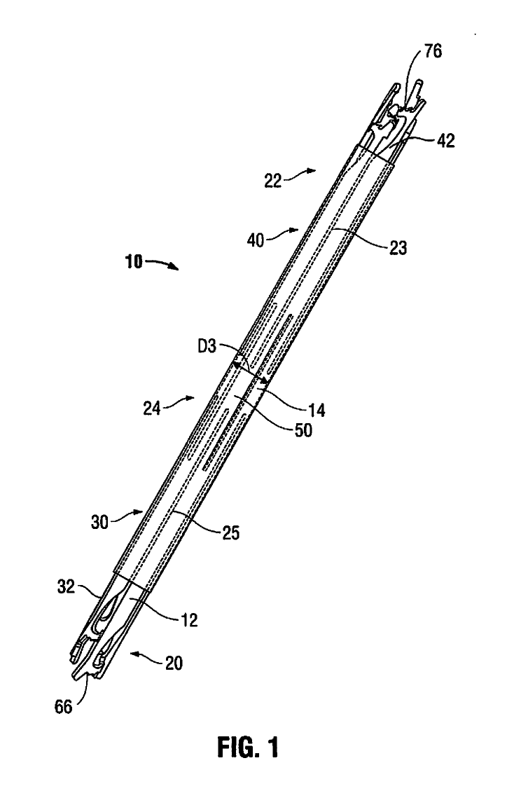

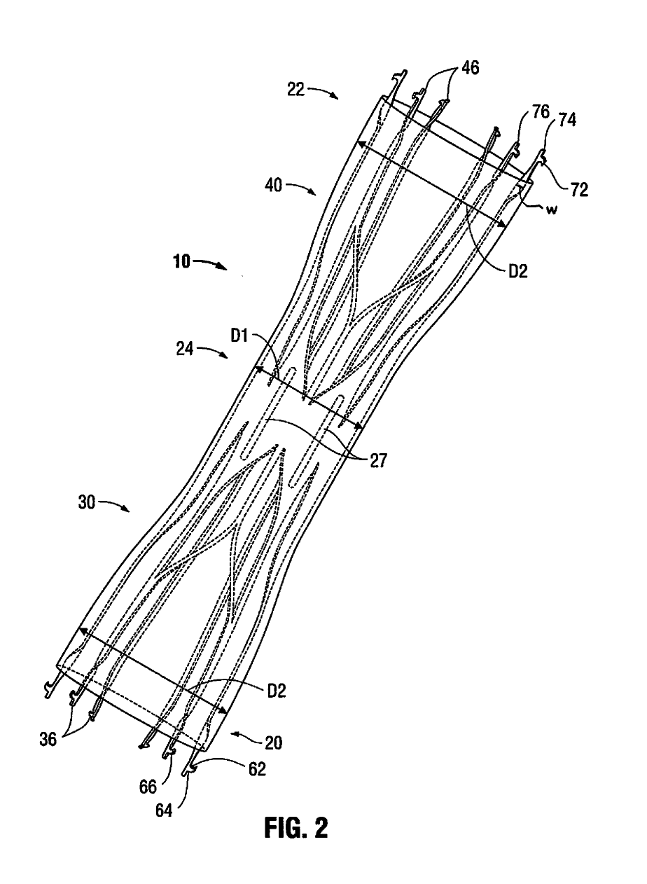

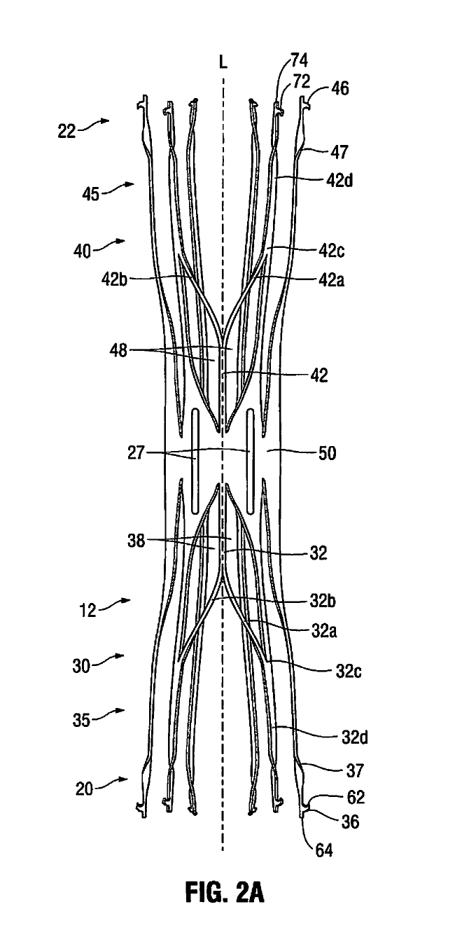

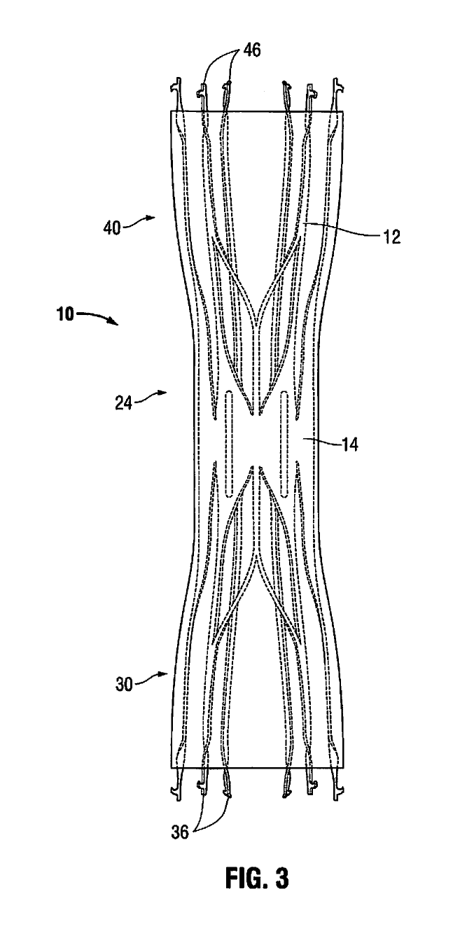

Referring now in detail to the drawings wherein like reference numerals identify similar or like components, FIGS. 1-3 illustrate one embodiment of the vascular device of the present invention, designated generally by reference numeral 10. FIG. 1 illustrates the device 10 in the collapsed delivery position (configuration) and FIGS. 2-3 illustrate the vascular device 10 in the expanded placement position (configuration). The vascular device 10 includes support structure 12 and a covering material 14. The covering material 14 can be composed of PTFE or other materials. With the covering material 14, the device 10 can in certain instances be considered as having a covered stent-like structure. The vascular device 10 has a reduced diameter intermediate portion to function as a choke point to reduce blood flow therethrough, thereby increasing blood flow to another vessel (or graft). Vascular device 10 can therefore be used to treat vascular access steal syndrome wherein too high blood flow in one vessel (or graft) is “stealing” necessary blood flow from another vessel (or graft), which can cause ischemia and its resulting adverse effects on the patient, including necrosis, paralysis and even loss of a body extremity such as a hand or foot.

The vascular device 10 has a first or proximal portion 20, a second or distal portion 22 and an intermediate portion 24. The terms proximal and distal as used herein refer to the direction of blood flow—blood flowing in the proximal to distal direction. In the illustrated embodiment, the vascular device 10 is symmetric, with the proximal portion 20 and distal portion 22 identical so proximal and distal are used for ease of discussion as the device 10 can be placed in either direction. In alternate embodiments, the device can be asymmetric.

As shown, the proximal and distal portions 20, 22 expand to a greater amount than the intermediate portion 24. This creates a substantially hourglass shape to reduce blood flow as the blood enters the reduced diameter intermediate portion 24 which acts as a choke point. That is, due to the narrower intermediate portion 24, blood flow will be restricted and forced to another vessel. Thus, the device 10 can be implanted in a minimally invasive manner as described below to increase blood flow to another vessel. Note the flow is restricted but not fully cut off so blood can continue to flow downstream through the device 10.

The reduced intermediate portion 24 can be appreciated by reference to FIG. 2 wherein in the expanded configuration, the intermediate portion 24 has a diameter or transverse dimension D1 and the proximal portion 20 and distal portion 22 have a diameter or transverse dimension D2 which is greater than D1. Note that the proximal and distal portions 20, 22 can have the same dimension D2, or alternatively have different dimensions. In either case, their dimensions would exceed the dimension D1 of the intermediate portion 24.

The collapsed configuration of vascular device 10 reduces the overall profile to facilitate delivery to the site. The vascular device 10 is movable from a low profile collapsed configuration (condition) to facilitate insertion through the delivery sheath to a larger expanded placement configuration (condition) to enable atraumatic engagement with the vessel (or graft) walls to secure (mount) the vascular device 10 within the vessel or graft. In some embodiments, in the low profile reduced dimension configuration, the transverse dimension of the device 10 can be uniform along its length and when the device 10 expands, the proximal and distal portions 20, 22 can expand to a greater degree than the amount the intermediate portion 24 expands. This is shown in the Figures. The diameter or transverse dimension D2 of the device 10 in the expanded placement configuration is greater than the diameter or transverse dimension D3 of the device in the collapsed (delivery) configuration (The diameter or transverse dimension of the intermediate portion 24 is also greater in the expanded configuration (D1) than in the collapsed configuration (D3)). Alternatively, the transverse dimensions in the collapsed position can be different, e.g., the transverse dimension of the intermediate portion 24 can be less than the transverse dimension of the proximal portion 20 and the distal portion 22 in the collapsed position, with the vascular device 10 still expanding to a configuration with a reduced dimension intermediate portion 24 between its two ends.

The vascular device 10 can also be considered as having two regions 30, 40 on opposing sides of the intermediate portion 24 or opposing sides of a midpoint of the vascular device 10, with first region 30 including proximal portion 20 at its proximal region and second region 40 including distal portion 22 at its distal region. Region 30 has a first series of struts extending in a first direction and region 40 has a second series of struts extending in a second opposing direction. The struts flare out as discussed below.

The vascular device 10, i.e., support structure 12, is preferably formed from a single tube. In a preferred embodiment, the device 10, i.e., support structure 12, is composed of shape memory material, such as Nitinol, a nickel titanium alloy, or elgiloy, with a shape memorized expanded configuration, however, other materials such as stainless steel are also contemplated. A plurality of cutouts 25 are formed in region 30 and a plurality of cutouts 23 are formed in region 40, preferably by laser cutting although other techniques are contemplated. In the illustrated embodiment, six elongated cutouts are formed in the first region 30 and in the second region 40, creating two pairs of six strips or struts 32, 42 of substantially uniform width separated by the cutouts 25, 23, respectively. The first set of struts 32 thus extends in a first direction from tubular portion 50 of intermediate portion 24 and the second set of struts 42 extends from tubular portion 50 in an opposing direction. Cutouts 27 are formed in tubular portion 50 to enable expansion of the tubular portion 50 when the device transitions from the collapsed position of FIG. 1 to the expanded configuration of FIG. 2.

FIGS. 2 and 2A illustrate the expanded placement configuration of the device 10. The covering material has been removed in FIG. 2A for ease of illustration and explanation. As noted above, support structure 12 of device 10 has a first set of struts 32 and a second set of struts 42, each flaring outwardly away from the central tubular region 50 in the expanded configuration. The struts 32, 42 each have a converging region respectively, transitioning into the tubular portion 50. At the opposing end, the struts 32, 42 each have a flared region 35, 45. The device 10 can expand to a diameter to accommodate the size of a vessel (or graft), applying a radial force against the vessel (or graft) wall to help maintain the device 10 in position. Diameters (or transverse dimensions) depend on the internal diameter of the vessel wall (or graft wall).

Struts 32 of region 30 are spaced apart as shown and extend at an angle away (outwardly) from the longitudinal axis L of device 10 to provide a flared region 35 at proximal portion 20. Preferably, this angle or taper is about 10 degrees, although other dimensions are contemplated. That is, the struts 32 extend outwardly away from the tubular portion 50 of the intermediate portion 24 in a proximal direction. Stated another way, region 30 extends from the flared region 35 toward the central longitudinal axis L of the device 10 and into tubular portion 50. Note that the struts 32 can in some embodiments initially extend longitudinally aligned with the tubular region 50 for a distance before angling outwardly away from the longitudinal axis. In other embodiments, they can angle outwardly from the tubular region 50 without initially extending a longitudinally aligned distance. For clarity, not all of the struts 32 (42) or sections of each strut 32 (42) are labeled in the drawings, it being understood that the non-labeled struts have the same configurations.

Struts 42 of region 40 in the illustrated embodiment, are identical to struts 32, but extend in an opposite direction. Struts 42 are spaced apart as shown and extend at an angle preferably about 10 degrees (other dimensions are also contemplated) away (outwardly) from the longitudinal axis L of device 10 to provide a flare. That is, the struts 42 extend outwardly away from the tubular portion 50 of the intermediate portion 24 in a distal direction. Stated another way, region 40 extends from the flared region 45 toward the central longitudinal axis L of the device 10 and into tubular portion 50. Note that the struts 42 can in some embodiments initially extend longitudinally aligned with the tubular region 50 for a distance before angling outwardly away from the longitudinal axis. In other embodiments, they can angle outwardly from the tubular region 50 without initially extending a longitudinally aligned distance. In the illustrated embodiment, when expanded, the six struts 32 and the six struts 42 are shown spaced approximately 60 degrees apart. It is also contemplated that a fewer or greater number of struts could be provided and spacing other than 60 degrees can be provided.

In the expanded placement configuration, a portion of each elongated strut 32, 42 has an outer surface respectively, for engagement with the vessel wall to retain the device 10 in position in the vessel. This region is angled with respect to the longitudinal axis. The outer surface of struts 32, 42 could be roughened to enhance engagement. Alternatively, a plurality of cutouts, atraumatic tabs, barbs or other penetrating members (not shown) can extend from the outer surface of one or more of the struts to engage the vessel wall to retain the vascular device 10.

Each of the struts 32, 42 can terminate in a hook 36, 46, respectively, which in the illustrated exemplary embodiment extends substantially perpendicular from the strut. This arrangement is achieved by torquing the struts 32, 42 at the respective region 37, 47 (or along an extended length of the strut) so the hook portions bend out of the plane. The hooks 36, 46 of device 10 lie in the plane of the connecting end strut region 37, 47 aligned with the width surface “w” of the region. The hooks can alternatively be formed or placed on fewer than all the struts. The hooks 36 include a penetrating tip 62 pointing in a direction toward the intermediate portion 24 and a heel 64 which extends proximally beyond penetrating tip 62. The penetrating tip 62 extends about a curved wall. The penetrating tip 62 in the illustrated embodiment extends substantially parallel to a longitudinal axis of the device 10. Opposite the curved wall on hook 36 are a plurality of teeth 66, with points or edges facing in a proximal direction, opposite the direction of the penetrating tip 62. Teeth 66 engage the vessel wall to provide additional retention to prevent movement of the implanted device in the proximal direction. Heel 64 extends past the tip 62 to function as a stop to prevent the strut portions from going through the vessel wall. For clarity, only some of the hooks and hook portions are labeled in the Figures.

The hooks 46 include a penetrating tip 72 pointing in a direction toward the intermediate portion 24 and a heel 74 which extends distally beyond penetrating tip 72. The penetrating tip 72 extends about a curved wall. The penetrating tip 72 in the illustrated embodiment extends substantially parallel to a longitudinal axis of the device 10. Opposite the curved wall on hook 46 are a plurality of teeth 76, with points or edges facing in a distal direction, opposite the direction of the penetrating tip 72. Teeth 76 engage the vessel wall to provide additional retention to prevent movement of the implanted device in the distal direction. Heel 74 extends past the hook 46 to function as a stop to prevent the strut portions from going through the vessel wall. For clarity, only some of the hooks and hook portions are labeled in the Figures.

In some embodiments, the hooks 36, 46 can be of the same size. In other embodiments the hooks 36 and 46 can be of different sizes. For example, hooks 36 can have a first set of hooks larger than a second set of hooks and hooks 46 can have a first set of hooks different than a second set of hooks. The hooks 36, 46 can be formed from a laser cut tube. Smaller hooks can be spaced axially inwardly with respect to the larger hooks to minimize the collapsed profile (transverse dimension) of the device when collapsed for insertion. In this embodiment, smaller hooks occupy the space created by the larger hooks so they can be considered as nesting within larger hooks. Alternatively, the struts can have a concave region which accommodates an adjacent hook. Some of the hooks 36 in the illustrated embodiment terminate at different distances from the tubular portion 50 (or midpoint of device 10) than other of the hooks 36, e.g., adjacent hooks, to facilitate collapse of the device 10 for delivery. Similarly, some of the hooks 46 can terminate at different distances (with respect to the tubular portion 50 or midpoint of the device 10) from other of the hooks 46, e.g., adjacent hooks.

The hooks or other vessel engaging structure can in alternate embodiments be placed on fewer than all the struts of the particular set of struts.

One or more of the struts 32, 42 of device 10 can be interconnected to enhance the stability of the device 10. More particularly, in the illustrated embodiment one of the struts 32 extends from tubular portion 50 and divides into two connecting struts or strut portions 32a, 32b (preferably of equal width) that angle way from each other (in different directions) to extend into an adjacent strut 32 at region 32c. This forms two closed geometric shapes 38. For clarity, not all of the identical parts are labeled in the drawings. In the illustrated embodiment, one of the six struts 32 bifurcates into two interconnecting struts, however, a different number of struts can divide into two connecting (interconnecting) struts or strut portions angling away from each other as in portions 32a, 32b to extend into an adjacent strut as in region 32c, and a different number of closed geometric shapes can be provided. Although preferably the struts 32 divide into connecting struts 32a, 32b of half the width of the undivided strut 32, the struts can bifurcate to form connecting struts of other dimensions.

After convergence of strut portions 32a, 32b at joining region 32c, it transitions into elongated mounting strut portions 32d which form flared mounting or anchoring region. The length of the strut portions 32d in the anchoring region can vary, with increased/decreased length increasing the flexibility/rigidity of the struts. The thickness of the strut portions can also vary to affect flexibility/rigidity.

Struts 42 extend from tubular portion 50 and in the illustrated embodiment one of the struts 42 divides into two connecting struts or strut portions 42a, 42b (preferably of equal width) that angle way from each other (in different directions) to extend into an adjacent strut 42 at region 42c. This forms two closed geometric shapes 48. For clarity, not all of the identical parts are labeled in the drawing. In the illustrated embodiment, one of the six struts 42 bifurcates into two connecting (interconnecting) struts, however, a different number of struts can divide into two connecting struts or strut portions angling away from each other as in portions 42a, 42b to extend into an adjacent strut as in region 42c, and a different number of closed geometric shapes can be provided. Although preferably the struts 42 divide into connecting struts 42a, 42b of half the width of the undivided strut 42, the struts can bifurcate to form connecting struts of other dimensions.

After convergence of strut portions 42a, 42b at joining region 42c, it transitions into elongated mounting strut portions 42d which form flared mounting or anchoring region. The length of the strut portions 42d in the anchoring region can vary, with increased/decreased length increasing the flexibility/rigidity of the struts. The thickness of the strut portions can also vary to affect flexibility/rigidity.

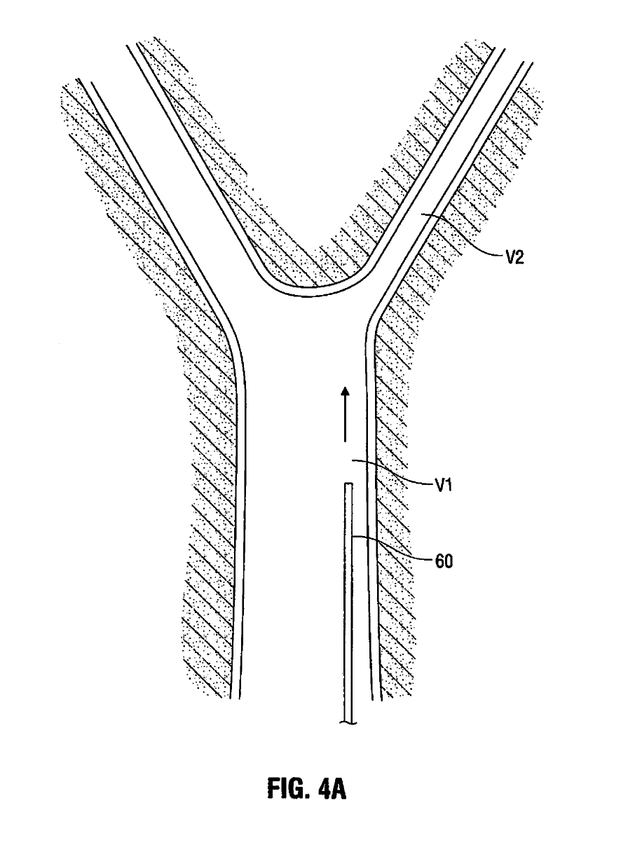

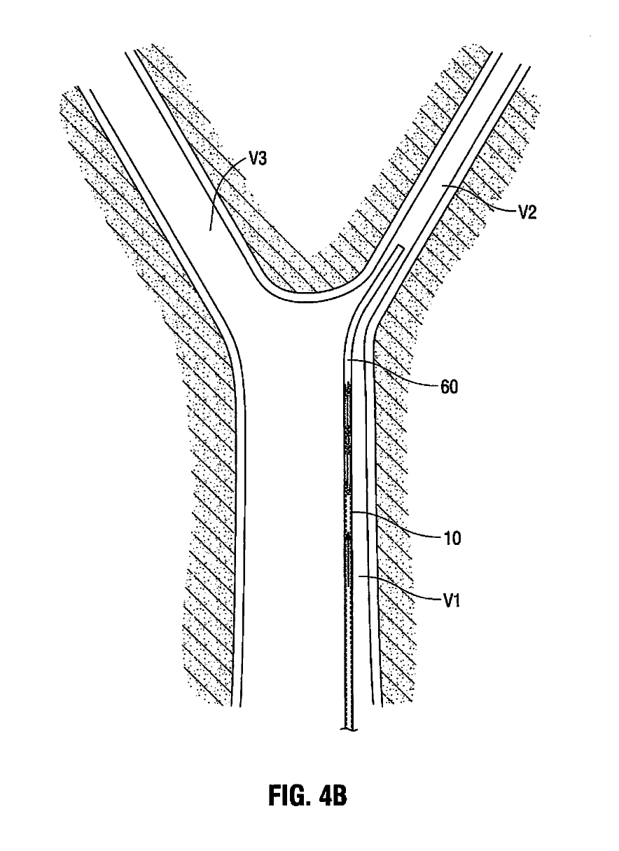

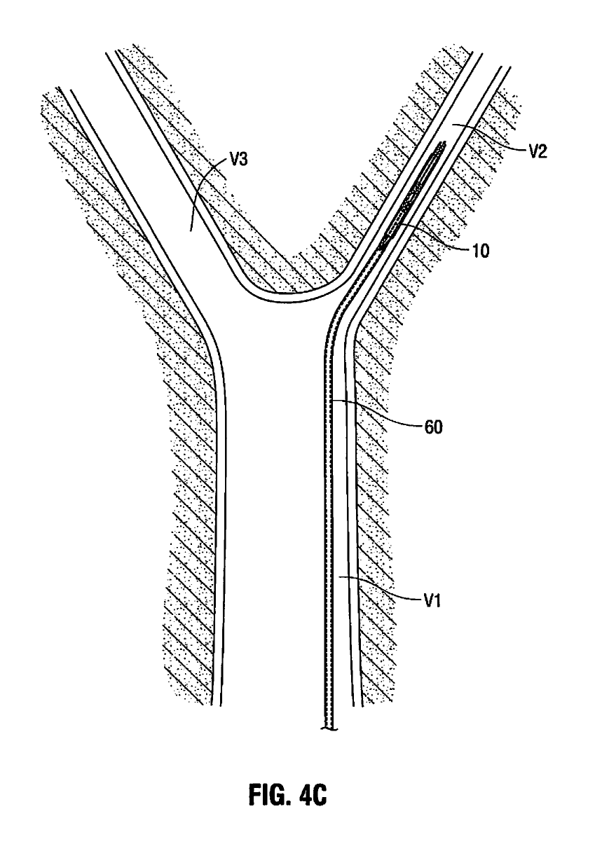

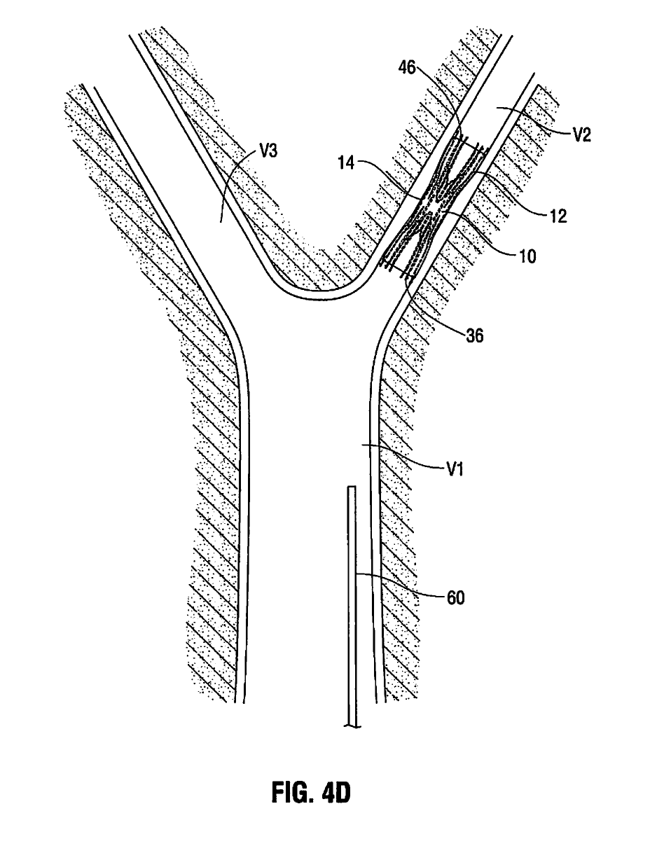

The device 10 in certain clinical applications can be placed in a branch vessel of a bifurcated vessel, just distal of the bifurcation or junction. Such placement reduces blood flow through the branch vessel, thereby automatically increasing blood flow through the other branch of the bifurcated vessel, providing additional blood flow to the region of the body more in need. It can also be placed within an arteriovenous fistula where blood flow is too high and causing vascular stealing syndrome. Such placement will reduce the flow through the AV fistula and automatically increase blood flow through the desired vessel. This placement of device 10 in the vessel is described in more detail below and illustrated in FIG. 4D. Thus, it can be appreciated, without the vascular device 10, blood flow of an amount X would flow in the first vessel and in an amount Y in the second vessel. With the placement of device 10 in the first vessel, the amount of flow in the first vessel would be reduced to X−Z while the amount of flow in the second vessel would be increased to Y+Z, with Z representing the amount of flow reduced in the first vessel and thereby increased in the second vessel.

The vascular device 10 of the present invention is described herein for use in a bifurcated vessel by way of example and its insertion method can be understood with reference to FIGS. 4A-4D. A delivery catheter or sheath 60 containing therein the vascular device 10 of FIG. 1 is inserted into the main vessel V1 and advanced into the first branch vessel V2. In certain applications, the sheath 60 can be inserted over a conventional guidewire (not shown). Once the sheath 60 is advanced through the main vessel V1 into the first branch vessel V2 (FIGS. 4B and 4C), the sheath 60 is withdrawn as in FIG. 4D, thereby freeing the vascular device 10 from the confines of the sheath 60 and allowing the vascular device 10 to expand to press the covering material 14 against the vessel wall. The vascular device 10 is preferably composed of shape memory material, such as Nitinol, that expands from a smaller configuration to its larger memorized configuration inside the body.

Upon full withdrawal of the sheath 60, leaving the vascular device 10 positioned as shown in FIG. 4D, the sheath 60 is removed from the patient, leaving the device 10 in the branching vessel V2. The covering material 14 helps to prevent blood flow through the struts 32, 42, thus keeping blood flow through the length of the vascular device 10 where it can be reduced as described herein. Blood flow is increased in branching vessel V3 (and reduced in branching vessel V2) due to the choke point formed by the reduced dimension intermediate portion as described above.

In alternate embodiments, instead of the automatic expansion of the vascular device 10 due to its shape memory material, the device is expanded by a conventional balloon catheter. A balloon catheter (not shown) is inserted within the sheath 60 so that the balloon underlies the vascular device 10. Inflation of the balloon radially expands the vascular device 10 against the wall of the vessel and then the balloon catheter is withdrawn.

It should be appreciated that the foregoing methods can be utilized to insert the vascular device 10 in a number of different branching vessel junctures.

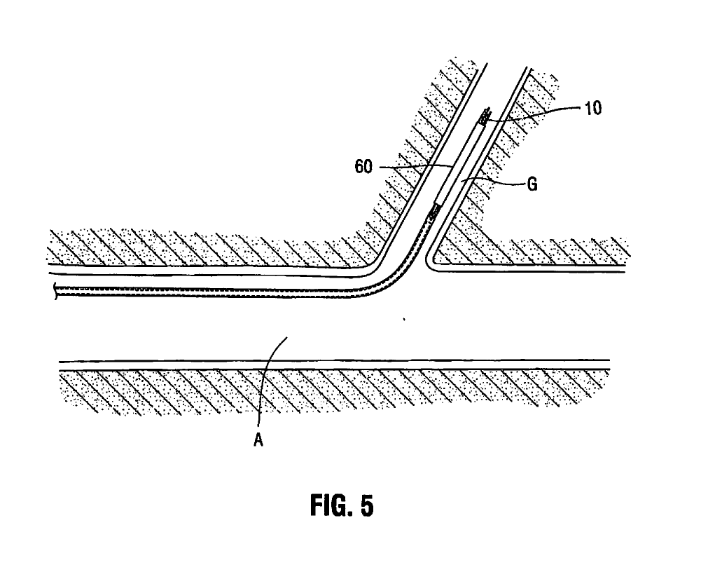

FIG. 5 illustrates the vascular device 10 utilized with an arteriovenous fistula wherein the device is implanted in a graft G connected to artery A. The device 10 is delivered by a delivery sheath 60′ into the graft G, and then removed to enable expansion of the device 10, thereby creating a choke point to decrease blood flow in graft G and increase blood flow in artery A.

In an alternate embodiment, the covered vascular device 10 has two layers of covering material—an outer layer and an inner layer. In such embodiments, the support structure 12 can either be embedded in the material layers or attached by various methods such as adhesive, overmolding or suture. When expanded, the outer layer will be sandwiched between the expanded device 10 and the vessel wall and the inner layer will contact the blood and prevent blood contact with support structure 12 of vascular device 10.

As can also be appreciated, even though covered vascular device 10 is shown with underlying support structure 12 and overlying material 14, respectively, the vascular device 10 can alternatively have the material on the inside or both the outside or inside as described above. Also, the covering material is shown to extend up to the hooks 36, 46 so the hooks are exposed. The covering material can also be of shorter length exposing more of the struts.

In yet another embodiment, the vascular device 10 can be used without a covering material and placed in the vessel. Although some blood flow might flow between the spaces within the struts 32, 42, the device 10, due to its reduced diameter intermediate portion 14, will still restrict blood flow.

While the above description contains many specifics, those specifics should not be construed as limitations on the scope of the disclosure, but merely as exemplifications of preferred embodiments thereof. For example, optionally multiple layers of material can be placed on the inside, outside or both the inside and outside of the vascular device. Also, the foregoing covered and uncovered vascular devices can be utilized in other vessels and grafts in addition to those described herein. Those skilled in the art will envision many other possible variations that are within the scope and spirit of the disclosure as defined by the claims appended hereto.

What is claimed is:

1. A vascular device for reducing blood flow in a first vessel or a first graft to enable increased blood flow in a second vessel or second graft, the vascular device comprising a support structure and a covering material and having a proximal portion, a distal portion and an intermediate portion, the support structure movable from a reduced profile insertion position to an expanded placement position, the covering material supported by the structure, the intermediate portion in the expanded position having a transverse dimension less than a transverse dimension of the proximal portion and the distal portion to restrict blood flow through the device and thereby increase blood flow to the second vessel or second graft.

2. The vascular device of claim 1, wherein the device has a substantially hourglass shape.

3. The vascular device of claim 1, further comprising a first plurality of penetrating members at the proximal portion and a second plurality of penetrating members at the distal portion.

4. The vascular device of claim 1, wherein the intermediate portion includes a tubular portion having a plurality of elongated slots formed therein.

5. The vascular device of claim 1, wherein the intermediate portion includes a tubular portion, and the support structure includes a first plurality of struts extending from and flaring out from the tubular portion in a distal direction and a second plurality of struts extending from and flaring out from the tubular portion in a proximal direction.

6. The vascular device of claim 5, wherein the first and second plurality of struts terminate in hooks.

7. The vascular device of claim 1, wherein the covering material is attached to an outer surface of the support structure.

8. The vascular device of claim 1, wherein the covering material is attached to an inner surface of the support structure.

9. The vascular device of claim 5, wherein at least one of the struts bifurcates to form connecting struts joining an adjacent strut.

10. The vascular device of claim 1, wherein the support structure is formed from a laser cut tube.

11. The vascular device of claim 1, wherein the support structure includes a first set of struts extending in a first direction and second set of struts extending in the opposite direction, the first and second set of struts extending from a tubular portion at the intermediate portion of the device.

12. A method of restricting blood flow in a first lumen and increasing blood flow in a second lumen, the method comprising:
providing a vascular device having an intermediate portion and a flared distal portion and a flared proximal portion, the intermediate portion having a reduced transverse dimension; and
placing the vascular device in the first lumen, the intermediate portion reducing blood flow by providing a choke point and thereby increasing blood flow to the second lumen.

13. The method of claim 12, wherein the first lumen is one of a vessel or a graft.

14. The method of claim 12, wherein the second lumen is one of a vessel or a graft.

15. The method of claim 12, further comprising the step of inserting a delivery sheath containing the vascular device and removing the delivery sheath to enable the vascular device to expand against the wall of the first lumen.

16. The method of claim 15, wherein the vascular device is made of shape memory material and the step of removing the sheath to enable the vascular device to expand causes the vascular device to automatically expand against the wall of the lumen.

17. The method of claim 15, further comprising the step of inserting a guidewire and inserting the delivery sheath over the guidewire.

18. A method of increasing blood flow in a vessel in an arteriovenous fistula, the method comprising:
providing a vascular device having an intermediate portion, a flared distal portion and a flared proximal portion, the intermediate portion having a reduced transverse dimension; and
placing the device in a lumen of a graft within a patient, the intermediate portion reducing blood flow in the lumen of the graft by providing a choke point and thereby increasing blood flow in a lumen of the vessel.

19. The method of claim 18, wherein the device includes a covering material contacting a wall of the graft.
